(19) United States
(12) Patent Application Publication
(10) Pub. No.: US 2021/0316010 A1
(43) Pub. Date: Oct. 14, 2021

(54) DRUG DELIVERY PRODUCT, COMPOSITION AND SYSTEM

(71) Applicant: MASTER DYNAMIC LIMITED, Hong Kong (CN)

(72) Inventor: Chia-Liang CHENG, Hong Kong (CN)

(73) Assignee: MASTER DYNAMIC LIMITED, Hong Kong (CN)

(21) Appl. No.: 17/266,889

(22) PCT Filed: Aug. 12, 2019
(86) PCT No.: PCT/CN2019/100255

§ 371 Date: Feb. 8, 2021

(30) Foreign Application Priority Data
Aug. 10, 2018 (HK) 18110301.3

(51) Int. Cl. A61K 47/69; A61K 47/64

(52) U.S. Cl. CPC A61K 47/6929; A61K 47/643

(57) ABSTRACT

A conjugate comprising (a) a nanodiamond; (b) human serum albumin (HSA) adhered on the surface of nanodiamond by physical adsorption; and (c) a drug molecule chemically linked to said human serum albumin, wherein said drug molecule has a therapeutic effect.

### TECHNICAL FIELD

The present invention relates to a drug delivery product, composition and system, in particular the present invention provides a drug delivery product, composition and system for the delivery of a drug to subjects with decreased toxicity side effects.

### BACKGROUND OF THE INVENTION

Chemotherapy is currently considered the most common method of treatment of cancer in the world, however drugs typically come with associated side effects.

In clinical practice, side effects are still a challenging problem in cancer therapy and treatment. The reduction of the side effects associated with drugs or therapy is an important issue, as this also encompasses the actual therapeutic effect of the drug.

Design of a drug delivery systems (DDS) can be utilised to solve or at least ameliorate some of these side effect issues. A DDS can include specific drug targeting/delivery, reduced toxicity whilst maintaining therapeutic effects, as well as the development of new and safer medicines.

Most of the advanced drug delivery systems (DDS) which have been developed have the purposes of improving the bioavailability of a drug product or active pharmaceutical ingredient (API), including by preventing or reducing premature degradation as well as enhancing drug uptake, which can be shown to maintain drug concentration within a requisite therapeutic window by controlling the drug release rate, which has been shown to reduce side effects by targeting diseased site and target cells.

Within the prior art, there have been many attempts and developments, for example targeted cancer therapy, by combining new or novel materials in order to carry and deliver anti-cancer drugs so as to minimize any side effects during delivery process of the drug.

Within the prior art, various nanostructured materials have been proposed for use in biology and medicine, for use in bio-imaging and also for drug delivery.

Within the prior art, nanoparticle-carriers are used in drug delivery for drug targeted transport and release of drugs and APIs.

It has been shown, for example, that the nanoparticles stimulate the endocytosis of drug resistant cells so as to raise intracellular drug concentration.

However, there has been concern with the use of delivery particles, such as nanoparticles, with issues pertaining to toxicity and accumulation within the body of a subject, as well as clumping together to such delivery particles, and thus the efficient loading of particles with drugs as well as effective and constant delivery to the body of a subject is required.

Further, a drug delivery system should be able to appropriately control the release and delivery of an API to a subject, so as to have a release profile which is appropriate for the particular application, for example providing a requisite blood plasma concentration to a subject for a therapeutic purpose.

Accordingly, in order to overcome such drawback of the prior art, to improve the quality of life of the subject, an improved drug delivery product, composition and system is needed.

### OBJECT OF THE INVENTION

It is an object of the present invention to provide drug delivery product, composition and system which overcomes or at least partly ameliorates at least some deficiencies as associated with the prior art.

### SUMMARY OF THE INVENTION

In a first aspect, the present invention provides conjugate comprising a nanodiamond, human serum albumin (HSA) adhered on the surface of nanodiamond by physical adsorption; and a drug molecule chemically linked to said human serum albumin, wherein said drug molecule has a therapeutic effect.

Preferably, said drug molecule is an anti-cancer drug molecule. The cancer may be a cancer of the lymph nodes, liver, lung, bone, kidney, brain, gastric, liver or colon tissues

The drug molecule may be doxorubicin hydrochloride C₂₇H₂₉NO₁₁ (DOX).

The drug molecule may be Methotrexate C₂₀H₂₂N₈O₅ (MTX).

Preferably, the nanodiamond has a size in the range of from 25 nm to 80 nm, more preferably in the range of from 35 nm to 65 nm, and more preferably the nanodiamond has a size of about 50 nm.

In a second, the present invention provides a pharmaceutical solution, comprising a plurality of a conjugates and a liquid carrier, wherein said conjugate comprises (a) a nanodiamond; (b) human serum albumin (HSA) adhered on the surface of nanodiamond by physical adsorption; and (c) a drug molecule chemically linked to said human serum albumin, wherein said drug molecule has a therapeutic effect.

Preferably, said drug molecule is an anti-cancer drug molecule. The cancer may be a cancer of the lymph nodes, liver, lung, bone, kidney, brain, gastric, liver or colon tissues

The drug molecule may be doxorubicin hydrochloride C₂₇H₂₉NO₁₁ (DOX).

The drug molecule may be Methotrexate C₂₀H₂₂N₈O₅ (MTX).

Preferably, the nanodiamond has a size in the range of from 25 nm to 80 nm, more preferably in the range of from 35 nm to 65 nm, and more preferably the nanodiamond has a size of about 50 nm.

In a third aspect, the present invention provides for the use of a conjugate for the manufacture of a medicament for the prevention or treatment of a subject, wherein said conjugate comprises (a) a nanodiamond, (b) human serum albumin (HSA) adhered on the surface of nanodiamond by physical adsorption; and (c) a drug molecule chemically linked to said human serum albumin, wherein said drug molecule has a therapeutic effect.

Preferably, said drug molecule is an anti-cancer drug molecule. The cancer may be a cancer of the lymph nodes, liver, lung, bone, kidney, brain, gastric, liver or colon tissues

The drug molecule may be doxorubicin hydrochloride (DOX).

The drug molecule may be Methotrexate C₂₀H₂₂N₈O₅ (MTX).

In a fourth aspect, the present invention provides a method of providing therapeutic treatment to a subject in need thereof, said method including the step of delivering to the subject a therapeutic amount of the pharmaceutical solution, wherein said pharmaceutical solution, comprises a plurality of a conjugates and a liquid carrier, wherein said conjugate comprises (a) a nanodiamond, (b) human serum albumin (HSA) adhered on the surface of nanodiamond by physical adsorption; and (c) a drug molecule chemically linked to said human serum albumin, wherein said drug molecule has a therapeutic effect.

Preferably, said drug molecule is an anti-cancer drug molecule. The cancer may be a cancer of the lymph nodes, liver, lung, bone, kidney, brain, gastric, liver or colon tissues

The drug molecule may be doxorubicin hydrochloride (DOX).

The drug molecule may be Methotrexate C₂₀H₂₂N₈O₅ (MTX).

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1(a) shows a UV-Visible light spectrum of ND-HAS-DOX conjugate at different pH values, i.e. pH7, pH8 and pH9;

FIG. 1(b) shows a graphical representation of the absorption of ND-HAS-DOX at different pH values, i.e. pH7, pH8 and pH9;

FIG. 1(c) shows a FTIR spectra of ND, ND-HAS, DOX, ND-DOX and ND-HAS-DOX;

FIG. 1(d) shows a graphical representation on the release rate of 50 ND-DOX at different pH values, i.e. pH6, pH7 and pH8;

FIG. 1(e) shows a graphical representation on the release rate of 50 ND-HAS-DOX at different pH values, i.e. pH6, pH7 and pH8;

FIG. 2(I) shows confocal images of SAS cell interaction with free DOX incubation for 4 hours;

FIG. 2(II) shows confocal images of SAS cell interaction with ND-HAS DOX complex incubation for 4 hours;

FIG. 3(a) shows a graphical representation of cell viability after treatment with free DOX and 50 ND-HAS-DOX with various concentration of DOX at 24 hours;

FIG. 3(b) shows a graphic representation of cell viability after treatment with free DOX and 50 ND-HAS-DOX with various concentration of DOX at 48 hours;

FIG. 3(c) shows a graphic representation of cell viability after treatment with free DOX and 50 ND-HAS-DOX with various concentration of DOX at 72 hours;

FIG. 4(a) shows a confocal image of MCTS incubated with DOX after 1 day;

FIG. 4(b) shows a confocal image of MCTS incubated with ND-DOX after 1 day;

FIG. 4(c) shows a confocal image of MCTS incubated with ND-HAS-DOX after 1 day;

FIG. 4(d) shows a confocal image of MCTS incubated with DOX after 2 days;

FIG. 4(e) shows a confocal image of MCTS incubated with ND-DOX after 2 days;

FIG. 4(f) shows a confocal image of MCTS incubated with ND-HAS-DOX after 2 days;

FIG. 5(I) shows an optical image of MCTS treated with ND, DOX and 50 ND-HAS-DOX for 4 days; and

FIG. 5(II) shows a graphical representation of the volume changes of MCTS during treatment.

### DETAILED DESCRIPTION OF THE INVENTION AND DRAWINGS

### 1. Invention

The present inventor has identified shortcomings of drug delivery systems, and have provided a provide drug delivery product, composition and system which has advantages of:
(a) Drug delivery carrier/particle having non-toxicity,
(b) Reduced particle aggregation,
(c) Increased drug loading to a delivery system;
(d) Provides for a release mechanism at a cancer site;
(e) Delivery and unloading of a drug compound or therapeutic agent at site specific treatment sites; and
(f) Improved release rate and consistency of delivery of the API to a subject.

In this context, the present inventor has utilised nanodiamond (ND) to develop a nanomedicine product which can reduce undesirable side effects as well as enhance the efficiency of drug delivery via use of acid sensitivity.

In accordance with the invention, human serum albumin (HSA) is adhered on the surface of nanodiamond (ND) by physical adsorption, and the HAS is chemically linked the anti-cancer drug doxorubicin hydrochloride (DOX) do as to form a conjugate.

Nanodiamond (ND) has been proposed as a means of delivering active pharmaceutical ingredients to a subject in need thereof, or for prophylactic purposes in some cases. Nano-diamond has been found to be non-toxic and biocompatible and as such, is considered both applicable and suitable for use in-vivo.

### 2. Use of Nanodiamond (ND)

Nanodiamond (ND) is a relatively new class of nanomaterial in the carbon families having excellent physical and chemical properties for these purposes for potential for use in a drug delivery system.

ND's spectroscopic signals (Raman and fluorescence) can be utilised for bio-labeling or imaging. The carbon sp3 nature of the ND lattice structure provides a unique Raman signal (˜1332 cm⁻¹) that is both strong and isolated.

The natural fluorescence of NDs from the crystal lattice defects, as well as by nanosize effect, further affords for another marker for bio imaging.

It has been shown that the biocompatibility of ND has been intensively investigated in the past few years, and ND's toxicity has been studied for different bio-systems.

The interaction of NDs with different kinds of cell cultures and biological tissues has been studied for pure/surface functionalized NDs with biomolecule conjugates. By taking advantage of ND's surface properties, an ND's surface can be functionalized with various molecular and ionic groups, followed by further conjugation with biomolecules of interest via physical adsorption or chemical linking, thus making ND an ideal platform for drug delivery.

Various methods for biomolecule or drug immobilization on ND are suggested and successful drug and gene delivery have been demonstrated [26-31] in the prior art.

However, nanoparticle aggregation, such as nanodiamond (ND) aggregation, can be problematic in drug delivery applications, especially for nanodiamonds with widths smaller than 50 nm.

This can be when the large surface area to volume ratio of nanodiamonds, whereby they tend to aggregate and form larger clustered particles, such to reduce the surface energy and capacity.

As such, these large aggregated nanodiamonds may not spread evenly onto the targeted area, and the contact area between nanodiamonds and the targeted area may be reduced and compromised.

This results in problems such as:
(i) a much higher drug dosage/amount is required to achieve the same effect of drugs with nanodiamond aggregation;
(ii) large aggregated nanodiamonds, by way of example, may not work properly as a diagnostic agent, as there is a high or increased chance for missing any positive results; and
(iii) the size of aggregated nanodiamonds may be too large for smooth flowing within the human's body fluid, and may in some cases compromise the safety to a subject.

### 3. Biological Aspects, Assays and Methodologies

It is known that for many decades, two-dimensional (2D) monolayer cell culture models have been used as a tool to drug metabolism, toxicity for the evaluation of the biological performance of nanoparticles.

Such a platform is easily handled, cost-effective, provides good reproducibility and the ability to grow a myriad of different cell types render 2D culture to be one of the most employed pre-clinical in vitro methodologies for chemotherapy development.

However, these 2D models have severe limitations, in that they lack ‘biomimicry’ and they are unable to provide three-dimensional (3D) cellular information that exists in vivo.

Further, such a 2D cultured cell is an aberrant gene and protein expression, which is caused by the stretched and undergo cytoskeletal rearrangements acquiring artificial polarity.

Recently, there are new pre-clinical methods, 3D tissue-like culture systems for detecting drug effect and nanotoxicity assessment. The 3D culture systems promote cancer cells growth alone with various cell types in a different method such as a scaffold, biochip, and spheroid, encouraging cell-cell and cell-matrix interactions that closely mimic the native environment of tumors. These interactions cause the 3D cultured cells to acquire morphological and cellular characteristics relevant to in vivo tumors.

In order to form a tumor, one characteristic of cancer cells is to form spheroids. The spheroids model, also-called multicellular tumor spheroid (MCTS), is similar to tissue but have no artificial substrate for helping cell attachment. They are formed in a liquid where coating the agarose on the culture flask prevents cell adhesion. Due to cell features and culture conditions, spheroids display various morphologies such as round, mass, grape-like, stellate.

There is much research in drug design and drug delivery using a MCTS model to demonstrate drug effect. MCTS is comprised of an innermost layer of necrotic cells with apoptotic cells in the peri-necrotic zone, surrounded by a middle layer of quiescent viable cells, and an outermost layer of highly proliferative and migratory cells.

Because the cell morphology is different to monolayer cell, the drug efficiency may be reduced. MCTS is a mimic model that can not only help to improve the chemotherapy drugs or development drugs, but advantageously also reduce the amount of animals required in an animal experimental model.

Scientists have demonstrated that the targeting of the drug target delivery via different methods such as pH responsive release drug, antibody conjugation. Human serum albumin (HSA), which is the most abundant protein of blood plasma with many important physiological functions has, was demonstrated to be able to target cancer via pH responsive release drug [42-45].

Studies have suggested a delivered drug to their targeting organs/tissue by binding with HSA [46, 47] Moreover, HSA also accounts for most of the antioxidant capacity of human serum, either directly or by binding and carrying radical scavengers. HSA not only against oxidation and influence the in vivo drug distribution but affect pharmacokinetic of drug [46]. Thus, HSA has been identified by the present inventor as having good potential substance for being the drug carrier.

### 4. Present Invention—Summary and Explanation

In accordance with the present invention as described and claimed, comparative studies are presented showing the efficiency of the ND-drug complex in the 2D- and 3D Human Oral Squamous Carcinoma cell (SAS) cellular model.

To characterize the ND-HAS-DOX conjugation, UV/Visible and FTIR spectroscopies were used. DOX release from ND-HSA-DOX at different pH was also measured.

The cellular uptake and penetration of ND, ND-drug can be measured by the laser confocal fluorescence image in 2D and 3D cellular model, to confirm detecting the colocalization of ND and DOX.

The cell viability test was performed using SAS cell line to compare the cytotoxic effect of DOX and ND-HSA-DOX complex.

The cytotoxic effect of DOX and ND-HSA-DOX complex was assessed in the 2D- and 3D-SAS cellular model via MTT assay, and multicellular tumor spheroid (MCTS) volume calculated.

The results showed pH-dependent drug release from ND-HSA-DOX complex is demonstrated, and ND-HSA-DOX is more efficient in the 3D-cultured cell compared to 2D-cultured cell.

Accordingly, the experimental studies support and demonstrate the potential that the acid sensitive ND-drug complex possess as a broad drug functionalization platform technology for nanoscale medicine as provided by the present invention, and the 3D cell model present real effects of drug delivery to tumor inside a human or animal body.

### 5. Synthesis, Characterization and Dispersion of ND-HSA, ND-HSA-DOX and ND-DOX

A study was conducted in accordance with the particulars of the present invention, using synthetic nanodiamond powders with average diameter of 50 nm sourced from Kay Diamond, USA which were purchased therefrom, after which carboxylation were used.

The ND was carboxylated according to methods described in detail such as in Chung P H, Perevedentseva E, Tu J S, Chang C C, Cheng C L: Spectroscopic study of bio-functionalized nanodiamonds. Diam Relat Mater 2006, 15(4-8):622-625 [48].

Before coating the anti-cancer drug doxorubicin hydrochloride (DOX) onto the NDs, to provide modification of ND's surface and avoid ND aggregation, Human Serum Albumin (HSA) was first adsorbed on ND surface.

The characterization of human serum albumin (HSA) adsorbed on ND surface, the UV-Visible spectra were used.

The absorption spectrum of HSA typically has an absorption band at 280 nm, corresponding to the absorption of UV light for three types of aromatic residues: (1) tryptophan (Trp), (2) phenylalanine (Phe) and (3) tyrosine (Tyr).

Among the three aromatic amino acids recited, the most intense absorption and emission is that of Trp, which has a higher molar absorptivity and intrinsic fluorescence quantum yield than both tyrosine and phenylalanine [49].

It has been demonstrated that HSA has been physically adsorbed on the surface of nanodiamond, whereby the mechanism of HSA adsorbed on nanodiamond is caused by hydrophobic attraction, hydrogen bonding, and ionic attraction Lee J W, Lee S, Jang S, Han K Y, Kim Y, Hyun J, Kim S K, Lee Y: Preparation of non-aggregated fluorescent nanodiamonds (FNDs) by non-covalent coating with a block copolymer and proteins for enhancement of intracellular uptake. Mol Biosyst 2013, 9(5):1004-1011 [50].

The net negative charge between HSA and the negatively charged ND surface may induce repulsive interaction therebetween, but the hydrophobic interaction and hydrogen bonding can induce the attractive interaction between them [51, 52].

50 nm ND particles size are bigger than 1 um (around 2100 nm) without conjugating HSA. The 50 ND-HSA complex decreased significantly to average size about 144 nm. The ζ-potential show that 50 nm ND has negative charge on surface −22 mV. Anionic amino acid residues cause HSA to has negative charge at neutral pH environment [53, 54].

After conjugating with HSA, the ζ-potential of 50ND-HSA is around −15 mV ND showed less negative ζ-potential, which is quite reasonable. Five samples of ND-HSA were prepared in the same time, every day, each sample was taken to measurement for 5 days.

The size and surface charge of ND-HSA complex is stably maintained with the size for 5 day, so the degree of aggregation is low and well dispersed ND solution was achieved.

After confirming the ND-HSA can be stable in phosphate buffered saline (PBS), ND-HSA were conjugated with DOX in PBS buffer. The DOX which was not adsorbed on nanodiamond was washed away (by twice centrifugation and washing with PBS).

The ND-HSA-DOX complex has been precipitated by centrifugation. In order to compare the function of HSA, ND-DOX was also prepared via the same method, but without HSA treatment.

After conjugation with DOX, the size and C potential of ND, ND-DOX, ND-HSA-DOX were determined. The average size of 50 ND, 50 ND-HSA was found to be around 2100 nm and 144 nm.

After conjugation with DOX, the size of ND, ND-HSA closely increased to 2966 nm and 155 nm respectively in PBS. The ζ-potential of 50ND-HSA-DOX and 50 ND-DOX were found to be around −15 mV With binding HSA, ND-HSA-DOX, it is still sufficiently small enough to be uptaken through the endocytotic pathway [55].

### 6. Loading and Release of Doxorubicin Hydrochloride (DOX)

Within utilizing the HAS intermediate in accordance with the present invention, the DOX adsorption on the ND surface may utilize the electrostatic interaction between carboxylic acid groups on ND and protonated amines on the DOX molecules.

However, DOX conjugation with the ND-HSA complex are used chemically linked, because HSA contains the cysteine 34 structure, which help HSA conjugate to DOX [49, 56].

The DOX loadings on the ND-HSA complex and drug loading efficiencies can be affected by different pH conditions. UV-Visible spectra was utilised to analyze the optimization of drug loading.

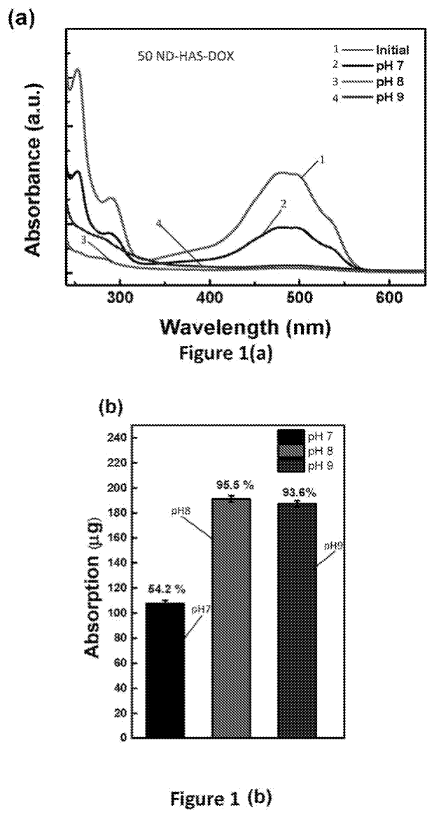

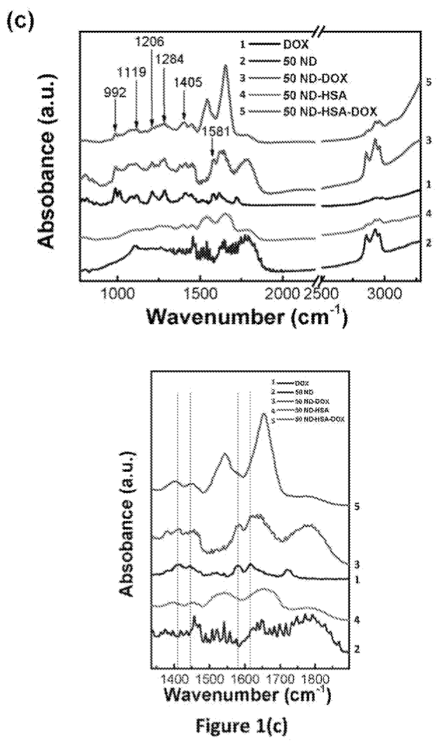

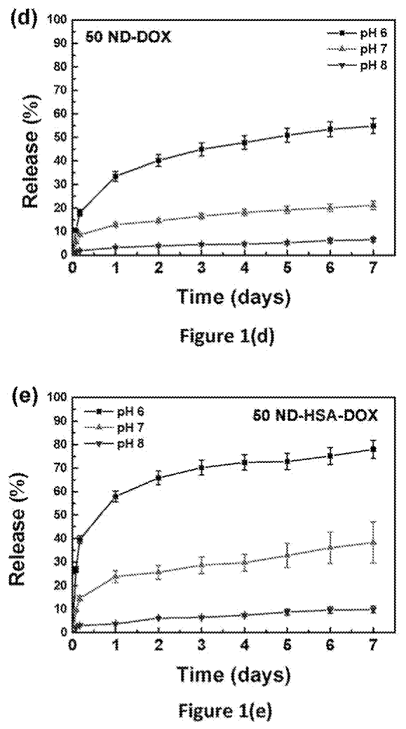

As shown in FIG. 1(a), it is demonstrated that DOX can have different loading efficiency at various pH values of a PBS solution.

DOX loading was respectively estimated to be 107.4 μg (pH 7), 191.2 μg (pH 8) and 187.4 μg (pH 9) accordingly.

Referring to FIG. 1(b), the ratio of DOX loading was shown to be 54.2%, 95.5% and 93.6% at pH 7, 8 and 9 respectively. The adsorbed DOX was determined through converting the UV-Visible absorbance to concentration using linear regression.

Thus, these FIGS. 1(a) and (b) display the drug loading and loading efficiency were achieved through leveraging the pH responsive properties of ND-HSA complex. ND-HSA has been shown to exhibit pH responsive drug adsorption properties.

So as to confirm the surface chemistry of the DOX, ND, ND-HSA, ND-DOX, ND-HSA-DOX complexes, these were characterized by FTIR spectra.

FIG. 1(c) represented the FTIR spectra are complicated and the most intense peaks are positioned in the range 1000-1700 cm⁻¹. The vibrational spectrum of DOX revealed C—O stretching of alcohol groups (1072, 1119, and 1206 cm⁻¹) in-plane bending of N—H (1612 and 1581 cm⁻¹), stretching of C—C (1405 cm⁻¹), stretching of C—O—C (1284 and 992 cm⁻¹) [57]. The DOX release profiles of ND-DOX and ND-HSA-DOX were evaluated by dialysis against PBS solution at pH 6.0, 7.0, and 8 as shown in FIG. 1(d) and FIG. 1(e) respectively.

It is apparent from the data that the DOX release profiles of ND-DOX and ND-HSA-DOX are pH-dependent, and all pH values have similar results.

So as to confine within the margin of beneficial effects through the slow release of DOX by ND-HSA-DOX complex via acid sensitive, when the ND or ND-HAS were loaded with anticancer drug DOX these were released to synergistically kill cancer cells.

Intracellular trafficking and distribution of DOX within SAS cells were used to further investigate the outcomes, by using confocal laser scanning microscopy (CLSM), as shown in the images of FIGS. 21 and 21I.

As is shown, the cell nuclei were stained with Hoechst 33324 which emits blue fluorescence (440 nm-484 nm) 202, and free DOX or DOX released from the ND-HSA-DOX complex which exhibited red fluorescence (565-620 nm) 204.

The 50 nm ND fluorescence was excited with 488 nm, and collected in range 500-515 nm. The ND's fluorescence in this range corresponds predominantly to emission from diamond H³ defect centers with emission peak near 505 nm, shown as green colour 206. The bright fields as evidenced, were also provided to reveal the morphologies of SAS cells.

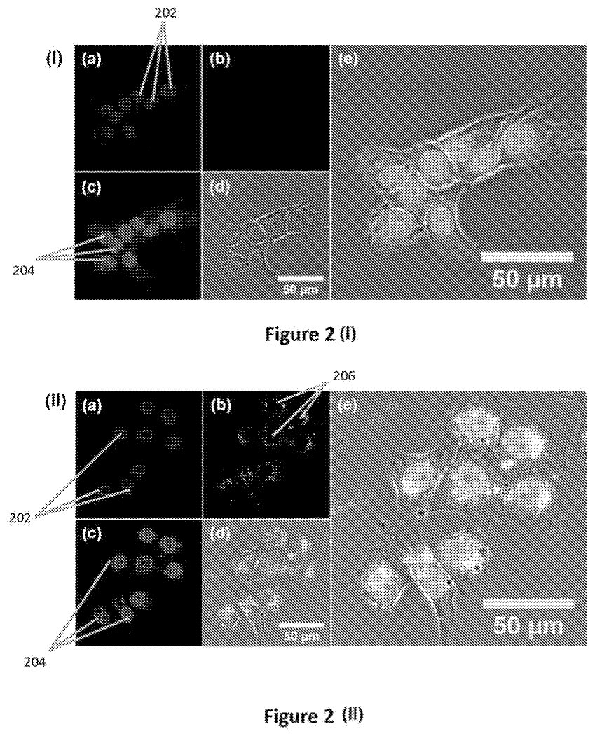

Following incubation for 4 hrs with the same dosage of DOX (20 μg/ml), as shown in FIG. 2 (I) was the control group, FIG. 2 (I)(b) showed no green fluorescence of ND.

The relatively red fluorescence 204 appeared in the cytoplasm as a few DOX phagocytized by SAS cells and was found with strong red fluorescence of DOX which began to appear in the cell nucleus as shown in FIG. 2 (I)(c).

However, a lot free DOX was distributed on cytoplasm, as free DOX distributes in the cell more homogeneously and gradually penetrated into nuclei, as shown in FIG. 2 (I)(e).

FIG. 2(II) shows the ND-HSA-DOX group, with the images of SAS interacting with ND-HSA-DOX as shown. FIG. 2 (II) (b) showed the green fluorescence of ND 206. The ND were observed to be localized in the cytoplasm and near the nuclei, but never penetrating into nuclei. This demonstrated that previously observed distribution of ND in the cell cytoplasm within the literature [17, 58].

As shown in FIG. 2 (II)(c) and FIG. 2 (II)-(e), the ND-HSA-DOX complex also is localized in the cytoplasm, the signals from DOX and ND do not observe co-localisation, and the strong red fluorescence 204 of DOX observed began to appear in the cell nucleus, with the ND only being near the nuclei, indicating that many ND-HSA-DOX had entered into the cells and much DOX was released from the ND-HSA-DOX complex.

The pathway of ND-drug in the Cellular, by Zhu H, Wang Y, Hussain A, Zhang Z P, Shen Y Y, Guo S R: Nanodiamond mediated co-delivery of doxorubicin and malaridine to maximize synergistic anti-tumor effects on multi-drug resistant MCF-7/ADR cells. J Mater Chem B 2017, 5(19):3531-3540, were proved, with the ND-drug complex indicating the formation of lysosomes, and the internalization of the ND-drug loading via an endosome/lysosome pathway, and the drug entered in the lysosomes or in the nuclei [59].

Importantly and in accordance with the present invention, these results demonstrated that the ND-HSA-DOX complex could enter the acidic environment of lysosomes to effectively release the anti-cancer drug DOX.

The red fluorescence of DOX at the ND-HSA-DOX group was found to be relatively stronger than that of free DOX groups, manifesting the enhanced uptake and release of DOX inside cells in the ND-HSA-DOX groups, and DOX was mainly distributed in the cell nuclei.

It is noted by the present inventor that Chan M S, Liu L S, Leung H M, Lo P K: Cancer-Cell-Specific Mitochondria-Targeted Drug Delivery by Dual-Ligand-Functionalized Nanodiamonds Circumvent Drug Resistance. ACS Appl Mater Interfaces 2017, 9(13):11780-11789, found that the majority localization of DOX is highly depended on its dosage, the DOX molecules are taken up by cells via endocytosis pathway and localized in lysosomes, when high dosage of DOX is used, some of them may be released from lysosomes, get into cytoplasm, and then enter the nuclear subsequently to kill tumor cells.

In the present study, it is proposed the modification of ND as carrier to deliver low dosage of DOX so to kill cancer cells instead of high dosage of DOX to do the same task. Thus, the results of the present study suggested that intracellular delivery of DOX could be regulated via ND-HSA complex platform, in accordance with the present invention.

A publication in the literature reported that free DOX has been located in nuclear of HeLa cells after treatment for 5 h, Li Y Q, Zhou X P, Wang D X, Yang B S, Yang P: Nanodiamond mediated delivery of chemotherapeutic drugs. J Mater Chem 2011, 21(41):16406-16412 [60], and from FIG. 2 (II) that the ND-HSA-DOX treatment for 4 h DOX was mainly distributed more focussedly in the cell nuclei, as expected, whereby the present invention is shown to be highly helpful to transport chemotherapeutic agents that are not cell permeable and directed them to specific intracellular organelles via such a ND-HSA delivery system.

Thus and importantly, the present study supports the present invention in that is demonstrated that DOX could be effectively delivered into cancer cells by ND-HSA-DOX complex for chemotherapy.

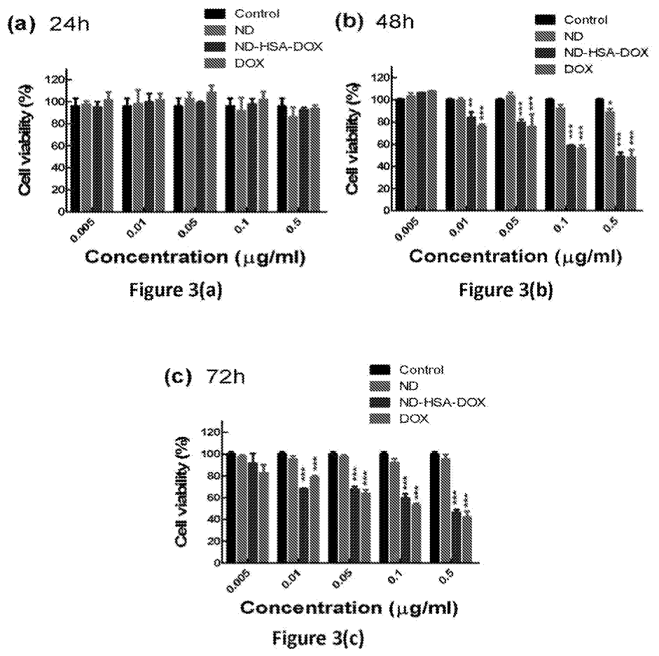

Referring to FIGS. 3(a) to (c), FIG. 3(a) shows a graphical representation of cell viability after treatment with free DOX and 50 ND-HAS-DOX with various concentration of DOX at 24 hour; FIG. 3(b) shows a graphic representation of cell viability after treatment with free DOX and 50 ND-HAS-DOX with various concentration of DOX at 48 hour; and FIG. 3(c) shows a graphic representation of cell viability after treatment with free DOX and 50 ND-HAS-DOX with various concentration of DOX at 72 hour.

The 2D monolayer cells grown on flat plastic or glass surfaces do not reflect the essential physiology of real tissue, as in the human body the cells grow in a 3D environment.

To obtain more adequate and detailed information about ND-drugs interactions, the present inventor continued using a 3D MCST model as a means to evaluate efficacy in vitro, showing the 3Dculture can reduce the gap between cell cultures and living tissue, as it closely mimics the native environment of tumors.

In order to investigate the penetration of DOX with different formulations (ND-DOX and ND-HSA-DOX) in tissue model in vitro, SAS MCTS were used to incubate with ND, free DOX, ND-DOX and ND-HSA-DOX.

The ND and DOX with different formulations complex uptake and distribution in SAS MCTS model was monitored by using CLSM. It was found that the MCTS homogeneously and have a spherical and symmetrical shape with the dimension of about 400 μm in the culture medium.

The cell nuclei were stained with Hoechst 33324 which emits blue fluorescence (440 nm-484 nm), cell membrane were dyed with 3,3′-dipentyloxacarbocyanine iodide (DIOC'5), and the signal was collected in the 520-555 nm range, shown in red color.

DOX which exhibited cyan fluorescence (565-620 nm), the 50 nm ND fluorescence was excited with 488 nm and collected in range 500-515 nm, show green colour.

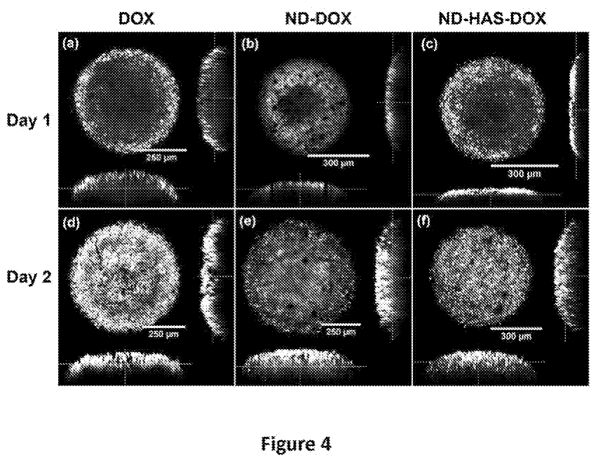

Referring to FIGS. 4(a) to 4(f) as was shown the X—Z and Y—Z confocal images of SAS MCTS incubated with DOX, ND-DOX, and ND-HSA-DOX group, whereby FIG. 4(a) shows a confocal image of MCTS incubated with DOX after 1 day; FIG. 4(b) shows a confocal image of MCTS incubated with ND-DOX after 1 day; FIG. 4(c) shows a confocal image of MCTS incubated with ND-HAS-DOX after 1 day; FIG. 4(d) shows a confocal image of MCTS incubated with DOX after 2 days; FIG. 4(e) shows a confocal image of MCTS incubated with ND-DOX after 2 days, and FIG. 4(f) shows a confocal image of MCTS incubated with ND-HAS-DOX after 2 days.

The penetration of ND and free DOX in the SAS MCTS were limited to the outer cell layers of the spheroids after 1 day of incubation time, although the strong fluorescence of ND-DOX and ND-HSA-DOX also appears in the outer cell layers of SAS MCTS after 1 day incubate. Furthermore, the weak fluorescence signal of ND and DOX arisen from the intermediate layers of the SAS MCTS indicated that some ND-DOX and ND-HSA-DOX complex can more effectively penetrate the spheroids.

### 7. Preparation of Nanodiamond, Nanodiamond-Doxorubicin and Nanodiamond-Albumin-Doxorubicin Complex

Interaction of doxorubicinhydrochloride (DOX) with nanodiamond was studied using diamond nanoparticles average diameter of 50 nm (Kay Diamond, USA) as-purchased and after surface modification were used. Detail treatment method of ND has been reported elsewhere.

In short, nanodiamond was treated with mixture of strong acids H₂SO₄:HNO₃ (1:3), to remove non-diamond admixtures and contaminations, and to modify the particles with COOH surface functional groups (carboxylated nanodiamond, cND) for further conjugation with desired molecules. Throughout the text, the ND means carboxylated nanodiamond.

7.1 ND-HSA

To avoid ND aggregation, Human Serum Albumin (HSA) was adsorbed on ND surface. For surface modification with adsorbed HSA, ND powders (50, 100 nm) of 2 mg in 900 ml double-distilled (D.D.) water, treated with ultra-sonication at power about 40 W for 5 min, were used.

Then 2 mg HSA powder (Sigma, USA) was added into 100 μl D.D. water, mixed with 50 nm ND solution and the solution was agitated for 2 hours at room temperature (Tᵣ).

After the agitation, the ND-HSA complex was centrifuged under 11,000 rcf for 10 min and the supernatant was removed. Then 1 ml D.D. water was added to disperse the 50 nm ND-HSA complexes.

7.2 ND-DOX

Doxorubicin hydrochloride was obtained from Sigma-Aldrich (USA). To prepare the complex, 5 mg of doxorubicin was first dissolved in 4 ml Dimethyl sulfoxide (DMSO, Sigma-Aldrich, USA).

Then doxorubicin was diluted to 400 μg/ml in 1 ml of standard phosphate buffer saline (PBS: NaCl 4 g; KCl 0.1 g, Na₂IPO₄ 0.72 g, KH₂PO₄ 0.21 g, H₂O 500 ml; pH 7.4). Moreover, 4 mg/ml of ND was added equivalently to the doxorubicin solution, obtaining its suspension with concentration of 2 mg/ml.

The mixture was thoroughly agitated for 2 h for better adsorption of the doxorubicin. After the agitation the mixture was centrifuged at the speed of 11,000 rcf at room temperature for 15 minutes to sediment the nanodiamond, including ND with adsorbed doxorubicin.

Then, 1 ml of PBS solution (pH value is 7.4) was added into the sediment, containing ND-doxorubicin complex.

The complex was subjected to weak ultrasound treatment to disaggregate the sediment and then to vortex for 30 minutes. The washing of ND-doxorubicin complex from solution to remove non-interacting doxorubicin was repeated 3 times.

### 8. Characterization of ND-HSA, ND-DOX and ND-HSA-DOX Complexes

The particle size and C potential were analyzed using the Zetasizer Nano ZS, from Malvern Instruments, Malvern, UK, with a 4 mW, 633 nm wavelength He—Ne laser on the base of dynamic light scattering method assembly, with a detection angle of 173°.

Nanodiamond, ND-HSA, DOX, ND-DOX and ND-HSA-DOX complex were diluted with PBS, to measure size and surface charge to obtain the concentrations.

After dilution to a concentration of 20 μg/ml, the pH was 7.4 at 25° C.

The pH values were measured using a SENTRON pH-meter, by Titan, Taiwan. For FTIR spectroscopic characterization, 20 μl of ND, DOX, ND-HSA, ND-DOX, ND-HSA-DOX complex solution each was placed on a Silicon substrate (1 cm×1 cm) and dried in air under room temperature.

FTIR spectroscopy, using a ABB Bomem MB 154 FTIR spectrometer, Switzerland, with a Deuterated Tri-Glycine Sulfate (DTGS) detector was used to obtain the sample's infrared spectra to confirm the forming of ND, DOX, ND-DOX and ND-HSA-DOX complex respectively at temperature 25° C. in air. The spectral resolution was 4 cm⁻¹.

### 9. UV-Visible Spectroscopic Analysis of ND-HSA, ND-DOX and ND-HSA-DOX Functionalization and Drug Loading Efficiency

The absorption spectra of the DOX solution before and after interaction with ND were measured using UV-Visible spectrometer JASCO V-550 by JASCO, US, at room temperature. The peak absorbance of DOX was found at 495 nm.

The intensity of adsorption peaks of the DOX were proportional to concentrations of the DOX in the solution, such that using a standard curve obtained by diluting from the specified DOX concentration and plotting absorbance at 495 nm, various concentration quantified the DOX concentration. Due to absorbance and drug concentration follow Beer-Lambert's law, the DOX concentrations after adsorption was converted by using the linear regression.

### 10. The pH Dependent Release of DOX from ND-DOX and ND-HSA-DOX Complex

The pH responsive release characteristics of the ND-DOX complexes were observed using PBS buffer of pH 6, 7 and 8.

After preparing the ND-DOX, the samples were resuspended in 1 mL of PBS and incubated at room temperature for an accumulated period of 2, 4, 24, 48 h in order to simulate in vitro drug release.

After incubation, the samples were centrifuged for 10 min at 11,000 rpm. Then, re-suspending the samples in fresh PBS for accumulating the remaining duration. The supernatants containing released DOX were collected for UV-Visible analysis.

### 11. Monolayer 2D SAS Cellular Uptake of ND, ND, DOX and ND-DOX Complex

Human Oral Squamous Carcinoma cell (SAS) cells were cultured in DMEM medium (Gibco, Invitrogen, UK). The medium was supplemented with 2 mM L-glutamine (Invitrogen, USA), 1.5 g/L sodium bicarbonate (Sigma, UK), 10% fetal bovine serum (Gibco/Life Technologies, Carlsbad, Calif., USA).

Cells were maintained under standard cell culture conditions in an incubator (Galaxy 170S, Eppendorf, USA) containing 95% air and 5% CO₂ at 37° C. humid environment.

Culture medium was replaced with a fresh medium every 48 or 72 hr. Cells were detached by treatment with 0.5% trypsin and 2.6 mM ethyl-enediaminetetraacetic acid (EDTA), from Gibco/Life Technologies, Carlsbad, Calif., USA, cultures were sub-cultured routinely at approximately 80% confluence.

The SAS cell (30,000 cells/well) was cultured on the 6 well contain coverslip for incubation of 2 days. Cells were treated with DOX and ND-HSA-DOX complex to observe their interaction.

Each sample was added to the medium, the sample concentration in the medium was 20 μg/ml, and cells were incubated together with the samples for 4 h.

Unreacted samples were removed by washing. The cells with DOX and ND-HSA-DOX complex adhered on the coverslips were fixed with 3.7% formaldehyde for 15 min and used for microscopic investigations. The emission of DOX was absorbed at 570-590 nm and ND was detected at 500-515 nm.

### 12. Cytotoxicity of ND-DOX Complexes in 2D Monolayer Cell Model

The effect of the ND, DOX and ND-DOX complexes on cell viability was determined by using MTT assay.

The MTT assay is a quantitative and rapid colorimetric method, based on the cleavage of a yellow tetrazolium salt to insoluble purple formazan crystals by the mitochondrial dehydrogenase of viable cells.

SAS cells were seeded in 96-well plates at the density of 5000 cells per well and incubated for 24 h to allow for cell attachment. Cells treated with blank vehicles were used as controls. Cells were treated with different concentrations of ND, DOX, ND-DOX complexes (10, 20, 30, 40 and 0.5 μg/ml) and the cells were incubated in the 5% CO₂ and 37° C. for 24 and 48 h. Upon completion of the incubation, stock MTT dye solution (20 μl, 5 mg/ml) was added to each well and the cells were incubated for another 4 h. The supernatant was removed and the formed MTT-formazan crystals were dissolved in 100 μl of DMSO and absorbance was recorded at 570 nm using a microplate reader. IC50 values were calculated and the optimum dose was used for further study.

### 13. SAS Multicellular Tumor Spheroids (MCTS) Formation and Growth Suppression Study

In order to promote the multicellular tumor spheroids (MCTS) formation, SAS cells were seeded in Gravity TRAP ULA Plate, by Insphero, at a density of 5,000 cells per well and cultured at 37° C. with 5% CO₂ for 3 days before drug treatment.

For the distributions of ND, DOX, ND-DOX and ND-HSA-DOX within MCTS were determined by confocal laser microscopy.

SAS MCTS were treated with ND, DOX, ND-DOX and ND-HSA-DOX for 4 days. Each day, one of treatment MCTS was harvested and fixed with 3.7% formaldehyde for 24 h. Washing the MCTS 3 time with PBS, and MCTS was incubated with DIOC'5 for 24 h. After washing 3 times, Hoechst 33342 was incubated with MCTS for 24 h. Then MCTS was observed by confocal microscopy.

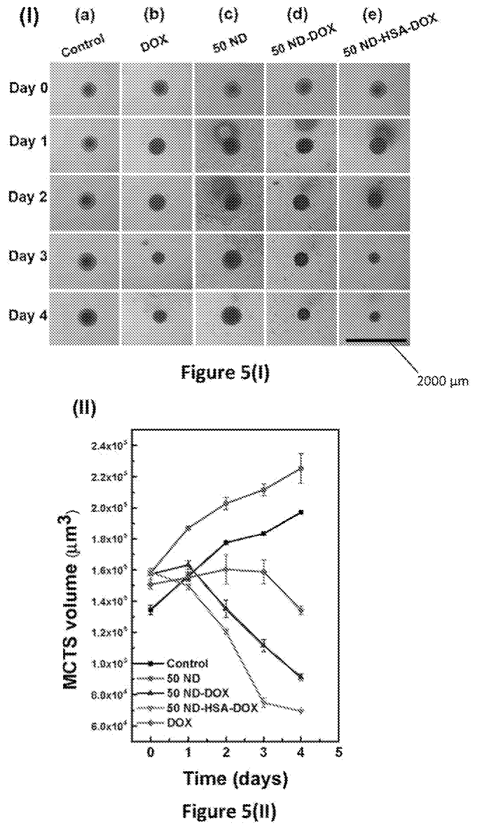

The growth inhibitory action of ND, DOX, ND-DOX complexes on MCTSs was measured. MCTSs with a diameter about 300 μm were co-cultured with each sample for 4 days. MCTSs were observed by dissecting microscope. The volume of MCTSs was calculated as:

$$V = \frac{( π \times a \times b )}{6}$$

where a represents the maximum diameter and b the minimum diameter of each MCTS. FIG. 5(I) shows an optical image of MCTS treated with ND, DOX and 50 ND-HAS-DOX for 4 days, and FIG. 5(II) shows a graphical representation of the volume changes of MCTS during treatment, whereby n=9 (three experiments repeated three times).

### 14. Statistical Analysis

The experimental results as analysed and described above were presented as mean±standard deviation (SD). Statistical difference between two groups was made using two-tailed Student's t-test. The P-value of <0.05 was considered statistically significant.

### 15. Anti-Cancer Compounds

The present invention has been described, in the experimental embodiments, using doxorubicin hydrochloride, C₂₇H₂₉NO_{H}, known as DOX. DOX is chemotherapy medication used to treat cancer. This includes breast cancer, bladder cancer, Kaposi's sarcoma, lymphoma, and acute lymphocytic leukemia. It is often used together with other chemotherapy agent, and the present invention is applicable to combination therapy.

As will be understood, other anti-cancer drugs may be used, such as Methotrexate Empirical formula: C₂₀H₂₂N₈O₅, known as MTX. Methotrexate is a chemotherapy agent and immune system suppressant. It is used to treat cancer, autoimmune diseases, ectopic pregnancy, and for medical abortions. Types of cancers it is used for includes breast cancer, leukemia, lung cancer, lymphoma, and osteosarcoma. Types of autoimmune diseases it is used for includes psoriasis, rheumatoid arthritis, and Crohn's disease. It can be given by mouth or by injection.

As will also be understood, the conjugate of the present invention may, in alternate embodiments, have more than one type of drug molecule attached thereto.

Further, in still further embodiments, the pharmaceutical solution may comprise a first plurality of conjugates with a first drug linked thereto, and a second plurality of conjugates with a second drug linked thereto.

The present invention, and embodiments thereof, is applicable to various cancer types, including lung cancer, colorectal cancer, gastric cancer, melanoma, pancreatic cancer, breast cancer, liver cancer and or prostate cancer.

### 16. References

1. Rahoui N, Jiang B, Taloub N, Huang Y D: Spatio-temporal control strategy of drug delivery systems based nano structures. J Control Release 2017, 255:176-201.
2. Zhang Y, Chan H F, Leong K W: Advanced materials and processing for drug delivery: the past and the future. Adv Drug Deliv Rev 2013, 65(1):104-120.
3. Zhu Q, Wu S D, Wu S S, Yang P, Sun J Z, Kang J B: Biodegradable Nano-Hybrid Vehicles for Combinatorial and Targeted Drug Delivery for Glioma Treatment. J Biomater Tiss Eng 2017, 7(5):363-370.
4. Xu H E, Jia F, Singh P K, Ruan S, Zhang H, Li X L: Synergistic anti-glioma effect of a coloaded nano-drug delivery system. Int J Nanomed 2017, 12:29-40.
5. Vaijayanthimala V, Lee D K, Kim S V, Yen A, Tsai N, Ho D, Chang H C, Shenderova O: Nanodiamond-mediated drug delivery and imaging: challenges and opportunities. Expert Opin Drug Del 2015, 12(5):735-749.
6. Perevedentseva E, Lin Y C, Jani M, Cheng C L: Biomedical applications of nanodiamonds in imaging and therapy. Nanomedicine-Uk 2013, 8(12):2041-2060.
7. Jiang K, Chi T, Li T, Zheng G R, Fan L L, Liu Y J, Chen X F, Chen S J, Jia L, Shao J W: A smart pH-responsive nano-carrier as a drug delivery system for the targeted delivery of ursolic acid: suppresses cancer growth and metastasis by modulating P53/MMP-9/PTEN/CD44 mediated multiple signaling pathways. Nanoscale 2017, 9(27):9428-9439.
8. Moreira A F, Dias D R, Costa E C, Correia I J: Thermo- and pH-responsive nano-in-micro particles for combinatorial drug delivery to cancer cells. Eur J Pharm Sci 2017, 104:42-51.
9. Liang X J, Meng H, Wang Y Z, He H Y, Meng J, Lu J, Wang P C, Zhao Y L, Gao X Y, Sun B Y et al: Metallofullerene nanoparticles circumvent tumor resistance to cisplatin by reactivating endocytosis. P Natl Acad Sci USA 2010, 107(16):7449-7454.
10. Mochalin V N, Shenderova O, Ho D, Gogotsi Y: The properties and applications of nanodiamonds. Nat Nanotechnol 2011, 7(1):11-23.
11. Ferrari A C, Robertson J: Origin of the 1150-cm(−1) Raman mode in nanocrystalline diamond. Phys Rev B 2001, 63(12).
12. Filik J, Harvey J N, Allan N L, May P W, Dahl J E P, Liu S, Carlson R M K: Raman spectroscopy of nanocrystalline diamond: An ab initio approach. Phys Rev B 2006, 74(3).
13. Kompan M E, Terukov E I, Gordeev S K, Zhukov S G, Nikolaev Y A: Photoluminescence spectra of ultradisperse diamond. Phys Solid State+1997, 39(12):1928-1929.
14. Iakoubovskii K, Stesmans A, Nouwen B, Adriaenssens G J: ESR and optical evidence for a Ni vacancy center in CVD diamond. Phys Rev B 2000, 62(24):16587-16594.
15. Lin Y C, Perevedentseva E, Tsai L W, Wu K T, Cheng C L: Nanodiamond for intracellular imaging in the microorganisms in vivo. J Biophotonics 2012, 5(11-12):838-847.
16. Lin Y C, Wu K T, Lin Z R, Perevedentseva E, Karmenyan A, Lin M D, Cheng C L: Nanodiamond for biolabelling and toxicity evaluation in the zebrafish embryo in vivo. J Biophotonics 2016, 9(8):827-836.
17. Huang K J, Lee C Y, Lin Y C, Lin C Y, Perevedentseva E, Hung S F, Cheng C L: Phagocytosis and immune response studies of Macrophage-Nanodiamond Interactions in vitro and in vivo. J Biophotonics 2017.
18. Zhu Y, Li J, Li W X, Zhang Y, Yang X F, Chen N, Sun Y H, Zhao Y, Fan C H, Huang Q: The Biocompatibility of Nanodiamonds and Their Application in Drug Delivery Systems. Theranostics 2012, 2(3):302-312.
19. Villalba P, Ram M K, Gomez H, Bhethanabotla V, Helms M N, Kumar A, Kumar A: Cellular and in vitro toxicity of nanodiamond-polyaniline composites in mammalian and bacterial cell. Mat Sci Eng C-Mater 2012, 32(3):594-598.
20. Blaber S P, Hill C J, Webster R A, Say J M, Brown L J, Wang S C, Vesey G, Herbert B R: Effect of Labeling with Iron Oxide Particles or Nanodiamonds on the Functionality of Adipose-Derived Mesenchymal Stem Cells. Plos One 2013, 8(1).
21. Ho D, Wang C H, Chow E K: Nanodiamonds: The intersection of nanotechnology, drug development, and personalized medicine. Sci Adv 2015, 1(7):e1500439.
22. Krueger A: New carbon materials: Biological applications of functionalized nanodiamond materials. Chem-Eur J 2008, 14(5):1382-1390.
23. Chao J I, Perevedentseva E, Chung P H, Liu K K, Cheng C Y, Chang C C, Cheng C L: Nanometer-sized diamond particle as a probe for biolabeling. Biophys J 2007, 93(6):2199-2208.
24. Krueger A, Lang D: Functionality is Key: Recent Progress in the Surface Modification of Nanodiamond. Adv Funct Mater 2012, 22(5):890-906.
25. Mochalin V N, Pentecost A, Li X M, Neitzel I, Nelson M, Wei C Y, He T, Guo F,
Gogotsi Y: Adsorption of Drugs on Nanodiamond: Toward Development of a Drug Delivery Platform. Mol Pharmaceut 2013, 10(10):3728-3735.
26. Zhu H, Wang Y, Hussain A, Zhang Z P, Shen Y Y, Guo S R: Nanodiamond mediated co-delivery of doxorubicin and malaridine to maximize synergistic anti-tumor effects on multi-drug resistant MCF-7/ADR cells. J Mater Chem B 2017, 5(19):3531-3540.
27. Ryu T K, Kang R H, Jeong K Y, Jun D R, Koh J M, Kim D, Bae S K, Choi S W: Bone-targeted delivery of nanodiamond-based drug carriers conjugated with alendronate for potential osteoporosis treatment. J Control Release 2016, 232:152-160.
28. Rammohan N, Filicko A, MacRenaris K, Ho D, Meade T J: Theranostic Nanodiamond-Gadolinium (III)-Doxorubicin Conjugates Improve Chemotherapy of Cancer Cells That Can Be Monitored by Magnetic Resonance Imaging. Int J Radiat Oncol 2016, 96(2):E589-E589.
29. Lim D G, Jung J H, Ko H W, Kang E, Jeong S H: Paclitaxel-Nanodiamond Nanocomplexes Enhance Aqueous Dispersibility and Drug Retention in Cells. Acs Appl Mater Inter 2016, 8(36):23558-23567.
30. Li L, Tian L, Wang Y L, Zhao W J, Cheng F Q, Li Y Q, Yang B S: Smart pH-responsive and high doxorubicin loading nanodiamond for in vivo selective targeting, imaging, and enhancement of anticancer therapy. J Mater Chem B 2016, 4(29):5046-5058.
31. Ryu T K, Lee G J, Rhee C K, Choi S W: Cellular Uptake Behavior of Doxorubicin-Conjugated Nanodiamond Clusters for Efficient Cancer Therapy. Macromol Biosci 2015, 15(10):1469-1475.
32. Chia S L, Tay C Y, Setyawati M I, Leong D T: Biomimicry 3D Gastrointestinal Spheroid Platform for the Assessment of Toxicity and Inflammatory Effects of Zinc Oxide Nanoparticles. Small 2015, 11(6):702-712.
33. Lee J, Lilly G D, Doty R C, Podsiadlo P, Kotov N A: In vitro toxicity testing of nanoparticles in 3D cell culture. Small 2009, 5(10):1213-1221.
34. Cukierman E, Pankov R, Stevens D R, Yamada K M: Taking cell-matrix adhesions to the third dimension. Science 2001, 294(5547):1708-1712.
35. Hamilton G: Multicellular spheroids as an in vitro tumor model. Cancer Lett 1998, 131(1):29-34.
36. Nath S, Devi G R: Three-dimensional culture systems in cancer research: Focus on tumor spheroid model. Pharmacol Ther 2016, 163:94-108.
37. Demut J, Grafe C, Hochhaus A, Clement J H: Multicellular spheroids: A model for nanoparticle-tumor cell interaction studies. Oncol Res Treat 2017, 40:273-273.
38. Demut J, Grafe C, Hochhaus A, Clement J H: Multicellular spheroids: A model for nanoparticle-tumor cell interaction studies. Oncol Res Treat 2016, 39:273-273.
39. Janko C, Hornung A, Pottler M, Friedrich R P, Zaloga J, Lyer S, Cicha I, Alexiou C: Cytotoxicity of unloaded and chemotherapeutics loaded nanoparticles in a multicellular 3D tumor spheroid model. N-S Arch Pharmacol 2015, 388:S83-S83.
40. Li L, Tian L, Zhao W J, Cheng F Q, Li Y Q, Yang B S: pH-sensitive nanomedicine based on PEGylated nanodiamond for enhanced tumor therapy. Rsc Adv 2016, 6(43):36407-36417.
41. Wang Y Y, Li P F, Chen F, Jia L Q, Xu Q H, Gai X M, Yu Y B, Di Y, Zhu Z H, Liang Y Y et al: A novel pH-sensitive carrier for the delivery of antitumor drugs: histidine-modified auricularia auricular polysaccharide nano-micelles. Sci Rep-Uk 2017, 7.
42. Kratz F: A clinical update of using albumin as a drug vehicle—A commentary. J Control Release 2014, 190:331-336.
43. Tao X, Jin S, Wu D, Ling K, Yuan L, Lin P, Xie Y, Yang X: Effects of Particle Hydrophobicity, Surface Charge, Media pH Value and Complexation with Human Serum Albumin on Drug Release Behavior of Mitoxantrone-Loaded Pullulan Nanoparticles. Nanomaterials (Basel) 2015, 6(1).
44. Mehtala J G, Kulczar C, Lavan M, Knipp G, Wei A: Cys34-PEGylated Human Serum Albumin for Drug Binding and Delivery. Bioconjugate Chem 2015, 26(5):941-949.
45. Kratz F, Warnecke A, Scheuermann K, Stockmar C, Schwab J, Lazar P, Druckes P, Esser N, Drevs J, Rognan D et al: Probing the cysteine-34 position of endogenous serum albumin with thiol-binding doxorubicin derivatives. Improved efficacy of an acid-sensitive doxorubicin derivative with specific albumin-binding properties compared to that of the parent compound. J Med Chem 2002, 45(25):5523-5533.
46. Yang F, Zhang Y, Liang H: Interactive Association of Drugs Binding to Human Serum Albumin. Int J Mol Sci 2014, 15(3):3580-3595.
47. Mariam J, Sivakami S, Dongre P M: Albumin corona on nanoparticles—a strategic approach in drug delivery. Drug Deliv 2016, 23(8):2668-2676.
48. Chung P H, Perevedentseva E, Tu J S, Chang C C, Cheng C L: Spectroscopic study of bio-functionalized nanodiamonds. Diam Relat Mater 2006, 15(4-8):622-625.
49. Kratz F, Warnecke A, Scheuermann K, Stockmar C, Schwab J, Lazar P, Druckes P, Esser N, Drevs J, Rognan D et al: Probing the cysteine-34 position of endogenous serum albumin with thiol-binding doxorubicin derivatives. Improved efficacy of an acid-sensitive doxorubicin derivative with specific albumin-binding properties compared to that of the parent compound. Journal of Medicinal Chemistry 2002, 45(25):5523-5533.
50. Lee J W, Lee S, Jang S, Han K Y, Kim Y, Hyun J, Kim S K, Lee Y: Preparation of non-aggregated fluorescent nanodiamonds (FNDs) by non-covalent coating with a block copolymer and proteins for enhancement of intracellular uptake. Mol Biosyst 2013, 9(5):1004-1011.
51. Chaves O A, Amorim A P D, Castro L H E, St Anna C M R, de Oliveira M C C, Cesarin-Sobrinho D, Netto-Ferreira J C, Ferreira A B B: Fluorescence and Docking Studies of the Interaction between Human Serum Albumin and Pheophytin. Molecules 2015, 20(10):19526-19539.
52. Yoon J Y, Park H Y, Kim J H, Kim W S: Adsorption of BSA on highly carboxylated microspheres—Quantitative effects of surface functional groups and interaction forces. J Colloid Interf Sci 1996, 177(2):613-620.
53. Kong X L, Huang L C L, Hsu C M, Chen W H, Han C C, Chang H C: High-affinity capture of proteins by diamond nanoparticles for mass spectrometric analysis. Anal Chem 2005, 77(1):259-265.
54. Ladam G, Gergely C, Senger B, Decher G, Voegel J C, Schaaf P, Cuisinier F J G: Protein interactions with polyelectrolyte multilayers: Interactions between human serum albumin and polystyrene sulfonate/polyallylamine multilayers. Biomacromolecules 2000, 1(4):674-687.
55. Zhang S L, Gao H J, Bao G: Physical Principles of Nanoparticle Cellular Endocytosis. Acs Nano 2015, 9(9):8655-8671.
56. Kratz F: Albumin as a drug carrier: Design of prodrugs, drug conjugates and nanoparticles. J Control Release 2008, 132(3):171-183.
57. Salaam A D, Hwang P T, Poonawalla A, Green H N, Jun H W, Dean D: Nanodiamonds enhance therapeutic efficacy of doxorubicin in treating metastatic hormone-refractory prostate cancer. Nanotechnology 2014, 25(42):425103.
58. Lin Y-C, Lin Z-R, Tsai L-W, Perevedentseva E, Karmenyan A, Cheng C-L: Spectral Analysis of Nanodiamond-Berberine Complex Interaction with Living Cells for Nanoparticle Mediated Drug Delivery. Journal of Biomedical Photonics & Engineering 2017, 3(1).
59. Chan M S, Liu L S, Leung H M, Lo P K: Cancer-Cell-Specific Mitochondria-Targeted Drug Delivery by Dual-Ligand-Functionalized Nanodiamonds Circumvent Drug Resistance. ACS Appl Mater Interfaces 2017, 9(13):11780-11789.
60. Li Y Q, Zhou X P, Wang D X, Yang B S, Yang P: Nanodiamond mediated delivery of chemotherapeutic drugs. J Mater Chem 2011, 21(41):16406-16412.

### 17. Invention Advantages

The present inventor has identified shortcomings of drug delivery systems, and provided a solution which has advantages of:

(a) Non-toxicity,

(b) Reduced particle aggregation, and

(c) Increased drug loading to a delivery system;

(d) Provides for a release mechanism at a cancer site;

(e) Delivery and unloading of a drug compound or therapeutic agent at site specific treatment sites; and

(f) Improved release rate and consistency of delivery of the API to a subject.

1. A conjugate comprising:
(a) a nanodiamond
(b) human serum albumin (HSA) adhered on the surface of nanodiamond by physical adsorption; and
(c) a drug molecule chemically linked to said human serum albumin, wherein said drug molecule has a therapeutic effect.

2. A conjugate according to claim 1, wherein said drug molecule is an anti-cancer drug molecule.

3. A conjugate according to claim 2, wherein the cancer is cancer of the lymph nodes, liver, lung, bone, kidney, brain, gastric, liver or colon tissues.

4. A conjugate according to any one of the preceding claims, wherein the drug molecule is doxorubicin hydrochloride, C₂₇H₂₉NO₁₁ (DOX).

5. A conjugate according to any one of claims 1 to 3, wherein the drug molecule is Methotrexate C₂₀H₂₂N₈O₅ (MTX).

6. A conjugate according to any one of the preceding claims, wherein the nanodiamond has a size in the range of from 25 nm to 80 nm.

7. A conjugate according to any one of the preceding claims, wherein the nanodiamond has a size in the range of from 35 nm to 65 nm.

8. A conjugate according to any one of the preceding claims, wherein the nanodiamond has a size of about 50 nm.

9. A pharmaceutical solution, comprising a plurality of a conjugates and a liquid carrier, wherein said conjugate comprises
(a) a nanodiamond;
(b) human serum albumin (HSA) adhered on the surface of nanodiamond by physical adsorption; and
(c) a drug molecule chemically linked to said human serum albumin, wherein said drug molecule has a therapeutic effect.

10. A pharmaceutical solution according to claim 9, wherein said drug molecule is an anti-cancer drug molecule.

11. A pharmaceutical solution according to claim 9 or claim 10, wherein the cancer is cancer of the lymph nodes, liver, lung, bone, kidney, brain, gastric, liver or colon tissues.

12. A pharmaceutical solution according to any one of claims 9 to 11, wherein the drug molecule is doxorubicin hydrochloride, C₂₇H₂₉NO₁₁ (DOX).

13. A pharmaceutical solution according to any one of claims 9 to 11, wherein the drug molecule is Methotrexate C₂₀H₂₂N₈O₅ (MTX).

14. A pharmaceutical solution according to any one of claims 9 to 13, wherein the nanodiamond has a size in the range of from 25 nm to 80 nm.

15. A pharmaceutical solution according to any one of claims 9 to 14, wherein the nanodiamond has a size in the range of from 35 nm to 65 nm.

16. A pharmaceutical solution according to any one any one of claims 9 to 15, wherein the nanodiamond has a size of about 50 nm.

17. The use of a conjugate for the manufacture of a medicament for the prevention or treatment of a subject, wherein said conjugate comprises:
(a) a nanodiamond
(b) human serum albumin (HSA) adhered on the surface of nanodiamond by physical adsorption; and
(c) a drug molecule chemically linked to said human serum albumin, wherein said drug molecule has a therapeutic effect.

18. The use according to claim 17, wherein said drug molecule is an anti-cancer drug molecule.

19. The use according to claim 18, wherein the cancer is cancer of the lymph nodes, liver, lung, bone, kidney, brain, gastric, liver or colon tissues.

20. The use according to any one of claims 17 to 19, wherein the drug molecule is doxorubicin hydrochloride, C₂₇H₂₉NO₁₁ (DOX).

21. The use according to any one of claims 17 to 19, wherein the drug molecule is Methotrexate C₂₀H₂₂N₈O₅ (MTX).

22. The use according to any one of claims 17 to 21, wherein the nanodiamond has a size in the range of from 25 nm to 80 nm.

23. The use according to any one of claims 17 to 22, wherein the nanodiamond has a size in the range of from 35 nm to 65 nm.

24. The use according to any one of claims 17 to 23, wherein the nanodiamond has a size of about 50 nm.

25. A method of providing therapeutic treatment to a subject in need thereof, said method including the step of delivering to the subject a therapeutic amount of the pharmaceutical solution, wherein said pharmaceutical solution, comprises a plurality of a conjugates and a liquid carrier, wherein said conjugate comprises
(a) a nanodiamond
(b) human serum albumin (HSA) adhered on the surface of nanodiamond by physical adsorption; and
(c) a drug molecule chemically linked to said human serum albumin, wherein said drug molecule has a therapeutic effect.

26. A method according to claim 25, wherein said drug molecule is an anti-cancer drug molecule.

27. A method according to claim 26, wherein the cancer is cancer of the lymph nodes, liver, lung, bone, kidney, brain, gastric, liver or colon tissues.

28. A method according to any one of claims 25 to 27, wherein the drug molecule is doxorubicin hydrochloride, C₂₇H₂₉NO₁₁ (DOX).

29. A method according to any one of claims 25 to 27, wherein the drug molecule is Methotrexate C₂₀H₂₂N₈O₅ (MTX).

30. A method according to any one of claims 25 to 29, wherein the nanodiamond has a size in the range of from 25 nm to 80 nm.

31. A method according to any one of claims 25 to 30, wherein the nanodiamond has a size in the range of from 35 nm to 65 nm.

32. A method according to any one of claims 25 to 31, wherein the nanodiamond has a size of about 50 nm.
